(19) United States
(12) Patent Application Publication
(10) Pub. No.: US 2023/0073963 A1
(43) Pub. Date: Mar. 9, 2023

(54) Synthesis of N-vinyl compounds by reacting cylic NH-compounds with acetylene in presence of homogenous catalyst

(71) Applicant: BASF SE, Ludwigshafen am Rhein (DE)

(72) Inventors: Thomas SCHAUB, Ludwigshafen am Rhein (DE); Elena Semina, Mannheim (DE); Pavel Tuzina, Ludwigshafen am Rhein (DE); Frank Bienewald, Ludwigshafen am Rhein (DE); A. Stephen K. Hashmi, Stuttgart (DE)

(73) Assignee: BASF SE, Ludwigshafen am Rhein (DE)

(21) Appl. No.: 17/757,697

(22) PCT Filed: Dec. 10, 2020
(86) PCT No.: PCT/EP2020/085396

§ 371 Date: Jun. 17, 2022

(30) Foreign Application Priority Data
Dec. 20, 2019 (EP) 19218545.2

(51) Int. Cl. C07D 265/10; B01J 31/02; B01J 23/46; C07D 207/267; C07D 211/76; C07D 225/02; C07D 239/88; C07D 241/44; C07D 263/22

(52) U.S. Cl. CPC C07D 265/10; B01J 31/0267; B01J 23/462; C07D 207/267; C07D 211/76; C07D 225/02; C07D 239/88; C07D 241/44; C07D 263/22

(57) ABSTRACT

A process can be used to produce N-vinyl compounds by homogeneous catalysis. In the process, acetylene is reacted with a cyclic compound having at least one nitrogen as a ring member, hearing a substitutable hydrogen residue (cyclic compound C), in a liquid phase in the presence of a ruthenium complex containing at least one phosphine as a ligand (RuCat).

Synthesis of N-vinyl compounds by reacting cylic NH-compounds with acetylene in presence of homogenous catalyst

Object of the invention is a process to produce N-vinyl compounds by homogeneous catalysis, wherein acetylene is reacted with a cyclic compound comprising a hydrogen substituted nitro gen as ring member in the liquid phase in the presence of a ruthenium complex comprising at least one phosphine as ligand.

From EP 512 656 it is known to produce vinyl compounds by reacting acetylene with a Bronsted acid in presence of a heterogeneous, supported catalyst comprising ruthenium.

EP-A 646571 discloses a homogeneously catalyzed reaction of acetylene with ammonia or a primary or secondary amino compound at 1 to 30 bars; 20 bars are used in the examples. Various catalysts are disclosed, inter alia catalysts based on ruthenium are mentioned.

WO 2006/056166 discloses a reaction of substituted alkynes with lactames, ureas or carba-mates which is catalyzed by a homogeneous catalyst. Acetylene is not included. As the substituted alkynes used are liquid, the reaction is performed at normal pressure.

In DE 19816479 a process for the synthesis of N-vinyl compounds by homogenous catalysis is described. An alkyne is reacted with ammonia or a primary or secondary amino compound in the liquid phase. Acetylene is mentioned but not used in the examples. Many suitable transition metal complexes are listed, but not the use ruthenium/phosphine complexes for reactions with acetylene.

As acetylene is gaseous, reactions with acetylene are usually performed under pressure. It is economic to keep the pressure as low as possible.

It was an object of this invention to provide a process for the synthesis of N-vinyl compounds, notably cyclic N-vinyl compounds, which can be performed at low pressure and wherein the N-vinyl compounds are obtained in high yield and selectivity.

Accordingly, the process above has been found.

To the Cyclic Compound

The cyclic compound is preferably a compound with a 5 to 8 membered ring system that comprises a cyclic compound having at least one nitrogen as ring member, bearing a substitutable hydrogen residue (cyclic compound C). Preferably, cyclic compound C has one or two nitrogen ring members bearing one or two substitutable hydrogen residues, more preferably one hydrogen residue.

In a particularly preferred embodiment, cyclic compound C is a cyclic amide, a cyclic urea or thiourea or a cyclic carbamate or thiocarbamate.

The cyclic amide comprises an amide group —NH—C(═O)—CH2- as element to the ring system.

The cyclic urea comprises a urea group —NH—C═(O)—NH— as element to the ring system.

The cyclic thiourea comprises a thiourea group —NH—C═(S)—NH— as element to the ring system.

The cyclic carbamate comprises a carbamate group —NH—C(═O)—O—as element to the ring sys tem.

The cyclic thiocarbamate comprises a thiocarbamate group —NH—C(═S)—O—or —NH—C(═O)—S—as element to the ring system.

The further carbon atoms of the ring system may be substituted or unsubstituted. Substituents to the carbon atoms may be, for example, carbonyl groups (═O), aliphatic or aromatic hydro-car bon groups that may comprise heteroatoms, notably oxygen in form of ether groups, two neighbored carbon atoms may be part of a further rings system, such as a cycloaliphatic or aromatic ring system.

In a most preferred embodiment, cyclic compound C is a cyclic amide.

The molecular weight of the cyclic compounds C is usually at maximum 1000 g/mol, preferably at maximum 500 g/mol.

Preferred Cyclic Amides are:

2-Pyrrolidone, 2-Piperidinone, Caprolactam, 8-Octanelactam, 2,3-Dihydro-1H-Isoindol-1-one, 2(1H)-Quinoxalinone, 4(3H)-Quinazolinone, 2,5-Piperazinedione, 2-Thiazolidinone, 2-Azabicyclo[2.2.1]hept-5-en-3-one

Preferred Cyclic Urea are:

2-Imidazolidinone, 4-Methyl-2-Imidazolidinone, 1,3-Dihydro-2H-Imidazol-2-one, 1,3-Dihydro-2H-Benzimidazol-2-one, 1,3-Dihydro-1-methyl-2H-Benzimidazol-2-one, 2,4-Imidazolidinedione, 5-Methyl-2,4-Imidazolidinedione, 5,5-Dimethyl-2,4-Imidazolidinedione, 5-Methyl-2,4(1H,3H)-Pyrimidinedione,

Preferred Cyclic Carbamate are:

2-Oxazolidinone, 4-Methyl-2-Oxazolidinone, 5-Methyl-2-Oxazolidinone, Tetrahydro-2H-1,3-Oxazin-2-one, 2(3H)-Benzoxazolone.

In the process of the invention, a cyclic compound C comprising a hydrogen substituted nitro gen as ring member is reacted with acetylene in the presence of at least one homogeneous Ru metal catalyst, having at least one phosphine as ligand (RuCat); also called vinylation catalyst hereinafter.

The vinylation catalyst RuCat of the process of the invention can be employed in the form of a preformed Ru metal complex which comprises the Ru metal compound and one or more ligands. Alternatively, the catalytic system is formed in situ in the reaction mixture by combining a Ru metal compound, herein also termed pre-catalyst, with one or more suitable ligands to form a catalytically active metal complex in the reaction mixture.

Preferred pre-catalysts are selected from neutral metal complexes, oxides and salts of ruthenium. Ruthenium compounds that are useful as pre-catalyst are, for example, [Ru(p-cymene)Cl2]2, [Ru(benzene)Cl2]n, [Ru(CO)2Cl2]n, [Ru(CO)3Cl2]2, [Ru(COD)(allyl)], [RuCl3·H2O], [Ru(acetylacetonate)3], [Ru(DMSO)4Cl2], [Ru(PPh3)3Cl2], [Ru(cyclopentadienyl)(PPh3)2Cl], [Ru(cyclopentadienyl)(CO)2Cl], [Ru(cyclopentadienyl)(CO)2H], [Ru(cyclopentadienyl)(CO)2]2, [Ru(pentamethylcyclopentadienyl)(CO)2Cl], [Ru(pentamethylcyclopentadienyl)(CO)2H], [Ru(pentamethylcyclopentadienyl)(CO)2]2, [Ru(indenyl)(CO)2Cl], [Ru(indenyl)(CO)2H], [Ru(indenyl)(CO)2]2, Ruthenocen, [Ru(2,2′-bipyridin)2(Cl)2·H2O], [Ru(COD)(Cl)2H]2, [Ru(pentamethylcyclopentadienyl)(COD)Cl], [Ru3(CO)12] and [Ru(tetraphenylhydroxycyclopentadienyl)(CO)2H].

For the vinylation of the process according to the present invention any complex ligands known in the art, in particular those known to be useful in ruthenium catalysed hydrogenations may be employed.

Suitable ligands of the catalytic system for the vinylation of the process according to the invention are, for example, mono-, bi-, tri- and tetra dentate phosphines of the formulae I and II shown below,

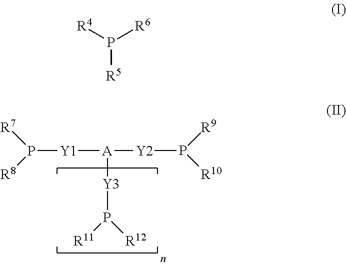

where
n is 0 or 1;

R4 to R12 are, independently of one another, unsubstituted or at least monosubstituted C1-C10-alkyl, C3-C10-cycloalkyl, C3-C10-heterocyclyl comprising at least one het-eroatom se lected from N, O and S, C5-C14-aryl or C5-C10-heteroaryl comprising at least one het-eroatom selected from N, O and S,

where the substituents are selected from the group consisting of: F, Cl, Br, OH, CN, NH2 and C1-C10-alkyl;

A is

i) a bridging group selected from the group unsubstituted or at least monosub-stituted N, O, P, C1-C6-alkane, C3-C10-cycloalkane, C3-C10-heterocycloalkane comprising at least one heteroatom selected from N, O and S, C5-C14-aromatic and C5-C6-heteroaromatic comprising at least one heteroatom selected from N, O and S,

where the substituents are selected from the group consisting of:

C1-C4-alkyl, phenyl, F, C1, Br, OH, OR16, NH2, NHR16 or N(R16)2,

where R16 is selected from C1-C10-alkyl and C5-C10-aryl;

or

ii) a bridging group of the formula (VI) or (VII):

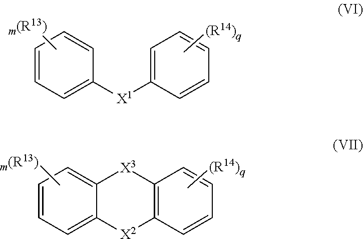

R13, R14 are, independently of one another, selected from the group C1 C10-alkyl, F, C1, Br, OH, OR15, NH2, NHR15 and N(R15)2,

where R15 is selected from 01-C10-alkyl and C5-C10-aryl;

X1, X2 are, independently of one another, NH, O or S;

X3 is a bond, NH, NR16, O, S or CR17R18;

R16 is unsubstituted or at least monosubstituted C1-C10-alkyl, C3 C10-cycloalkyl, C3-C10-heterocyclyl comprising at least one heteroatom se-lected from N, O and S, 05-C14-aryl or 05-C10-heteroaryl comprising at least one heteroatom selected from N, O and S,

where the substituents are selected from the group consisting of: F, C1, Br, OH, CN, NH2 and C1-C10-alkyl;

R17, R18 are, independently of one another, unsubstituted or at least monosub-stituted C1-C10-alkyl, C1-C10-alkoxy, C3-C10-cycloalkyl, C3-C10-cycloalkoxy, C3-C10-heterocyclyl comprising at least one heteroatom se-lected from N, O and S, C5-C14-aryl, C5C14-aryloxy or C5-C10-heteroaryl comprising at least one heteroatom selected from N, O and S,

where the substituents are selected from the group consisting of: F, C1, Br, OH, CN, NH2 and C1-C10-alkyl;

Y1, Y2, Y3 are, independently of one another, a bond, unsubstituted or at least monosubstituted methylene, ethylene, trimethylene, tetramethylene, pentameth-ylene or hexamethylene, where the substituents are selected from the group consisting of: F, C1, Br, OH, OR15, CN, NH2, NHR15, N(R15)2 and C1-C10-alkyl,

where R15 is selected from C1-C10-alkyl and C5-C10-aryl.

A is a bridging group. For the case that A is selected from the group unsubstituted or at least monosubstituted C1-C6-alkane, C3-C10-cycloalkane, C3-C10-heterocycloalkane, C5-C14-aromatic and C5-C6-heteroaromatic for the case (n=0), two hydrogen atoms of the bridging group are replaced by bonds to the adjacent substituents Y1 and Y2. For the case (n=1), three hydrogen atoms of the bridging group are replaced by three bonds to the adjacent substituents Y1, Y2 and Y3.

For the case that A is P (phosphorus), the phosphorus forms for the case (n=0) two bonds to the adjacent substituents Y1 and Y2 and one bond to a substituent selected from the group consisting of C1-C4-alkyl and phenyl. For the case (n=1), the phosphorus forms three bonds to the adjacent substituents Y1, Y2 and Y3.

For the case that A is N (nitrogen), the nitrogen for the case (n=0) forms two bonds to the adjacent substituents Y1 and Y2 and one bond to a substituent selected from the group consisting of C1-C4-alkyl and phenyl. For the case (n=1), the nitrogen forms three bonds to the adjacent substituents Y1, Y2 and Y3.

For the case that A is O (oxygen), n=0. The oxygen forms two bonds to the adjacent substituents Y1 and Y2.

Preference is given to complex catalysts which comprise at least one element selected from ruthenium and iridium.

In a preferred embodiment, the process according to the invention is carried out in the presence of at least one complex catalyst which comprises Ru and also at least one phosphorus do nor ligand of the general formula (II), where

n is 0 or 1;

R7 to R12 are, independently of one another, unsubstituted C1 C10-alkyl, C3-C10-cycloalkyl, C3-C10-heterocyclyl comprising at least one heteroatom selected from N, O and S, C5-C14-aryl or C5-C10-heteroaryl comprising at least one heteroatom se-lected from N, O and S;

A is

i) a bridging group selected from the group unsubstituted C1-C6-alkane, C3-C10-cycloalkane, C3-C10-heterocycloalkane comprising at least one heteroatom selected from N, O and S, C5-C14-aromatic and C5-C6-heteroaromatic comprising at least one heteroatom selected from N, O and S;

or

ii) a bridging group of the formula (VI) or (VII):

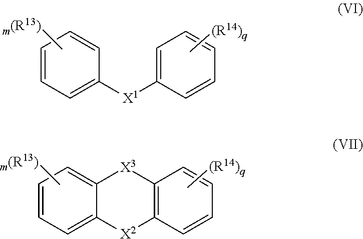

R13, R14 are, independently of one another, selected from the group C1 C10-alkyl, F, C1, Br, OH, OR15, NH2, NHR15 and N(R15)2,

where R15 is selected from C1-C10-alkyl and C5-C10-aryl;

X1, X2 are, independently of one another, NH, 0 or S;

X3 is a bond, NH, NR16, O, S or CR17R18;

R16 is unsubstituted C1-C10-alkyl, C3-C10-cycloalkyl, C3-C10-heterocyclyl comprising at least one heteroatom selected from N, O and S, C5-C14-aryl or C5-C10-heteroaryl comprising at least one heteroatom selected from N, O and S;

R17, R18 are, independently of one another, unsubstituted C1-C10-alkyl, C1 C10-alkoxy, C3-C10-cycloalkyl, C3-C10-cycloalkoxy, 03-C10-heterocyclyl com-prising at least one heteroatom selected from N, O and S, C5-C14-aryl, C5-C14-aryloxy or C5-C10-heteroaryl comprising at least one heteroatom selected from N, O and S;

Y1, Y2, Y3 are, independently of one another, a bond, unsubstituted methylene, ethylene, trimethylene, tetramethylene, pentamethylene or hexamethylene.

In a further preferred embodiment, the process according to the invention is carried out in the presence of at least one complex catalyst which comprises Ru and also at least one phosphorus donor ligand of the general formula (VIII),

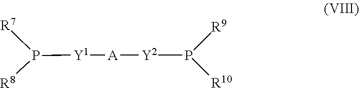

where

R7 to R10 are, independently of one another, unsubstituted or at least monosubstituted C1-C10-alkyl, C3-C10-cycloalkyl, C3-C10-heterocyclyl comprising at least one het-eroatom se lected from N, O and S, C5-C14-aryl or C5-C10-heteroaryl comprising at least one heteroatom selected from N, O and S,

where the substituents are selected from the group consisting of: F, C1, Br, OH, CN, NH2 and C1-C10-alkyl;

A is

i) a bridging group selected from the group unsubstituted or at least monosub-stituted N, O, P, C1-C6-alkane, C3-C10-cycloalkane, C3-C10-heterocycloalkane com-prising at least one heteroatom selected from N, O and S, C5-C14-aromatic and C5-C6-heteroaromatic comprising at least one heteroatom selected from N, O and S,

where the substituents are selected from the group consisting of:

C1-C4-alkyl, phenyl, F, C1, Br, OH, OR15, NH2, NHR15 or N(R15)2,

where R15 is selected from C1-C10-alkyl and C5-C10-aryl;

or

ii) a bridging group of the formula (VI) or (VII):

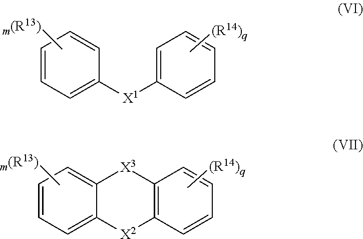

i) a bridging group selected from the group unsubstituted or at least monosub-stituted N, O, P, C1-C6-alkane, C3-C10-cycloalkane, C3-C10-heterocycloalkane com-prising at least one heteroatom selected from N, O and S, C5-C14-aromatic and C5-C6-heteroaromatic comprising at least one heteroatom selected from N, O and S,

where the substituents are selected from the group consisting of:

C1-C4-alkyl, phenyl, F, C1, Br, OH, OR15, NH2, NHR15 or N(R15)2,

where R15 is selected from C1-C10-alkyl and C5-C10-aryl;

or

ii) a bridging group of the formula (VI) or (VII):

m, q are, independently of one another, 0, 1, 2, 3 or 4;

R13, R14 are, independently of one another, selected from the group C1 C10-alkyl, F, C1, Br, OH, OR15, NH2, NHR15 and N(R15)2,

where R15 is selected from C1-C10-alkyl and C5-C10-aryl;

X1, X2 are, independently of one another, NH, 0 or S,

X3 is a bond, NH, NR16, O, S or CR17R18;

R16 is unsubstituted or at least monosubstituted C1-C10-alkyl, C3 C10-cycloalkyl, C3-C10-heterocyclyl comprising at least one heteroatom selected from N, O and S, C5-C14-aryl or C5-C10-heteroaryl comprising at least one heteroatom selected from N, O and S,

where the substituents are selected from the group consisting of: F, C1, Br, OH, CN, NH2 and C1-C10-alkyl;

R17, R18 are, independently of one another, unsubstituted or at least monosub-stituted C1-C10-alkyl, C1-C10-alkoxy, C3-C10-cycloalkyl, C3-C10-cycloalkoxy, 03-C10-heterocyclyl comprising at least one heteroatom se-lected from N, O and S, C5-C14-aryl, C5-C14-aryloxy or 05-C10-heteroaryl comprising at least one heteroatom selected from N, O and S,

where the substituents are selected from the group consisting of: F, C1, Br, OH, CN, NH2 and C1-C10-alkyl;

Y1, Y2 are, independently of one another, a bond, unsubstituted or at least mono-substituted methylene, ethylene, trimethylene, tetramethylene, pentameth-ylene or hexamethylene,

where the substituents are selected from the group consisting of: F, C1, Br, OH, OR15, CN, NH2, NHR15, N(R15)2 and C1-C10-alkyl,

where R15 is selected from C1-C10-alkyl and C5-C10-aryl.

In a further preferred embodiment, the process according to the invention is carried out in the presence of at least one complex catalyst which comprises at least one element selected from groups 8, 9 and 10 of the Periodic Table of the Elements and also at least one phosphorus do nor ligand of the general formula (IX),

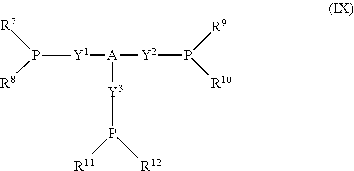

where

R7 to R12 are, independently of one another, unsubstituted or at least monosubstituted C1-C10-alkyl, C3-C10-heterocyclyl comprising at least one heteroatom selected from N, O and S, C5-C14-aryl or C5-C10-heteroaryl comprising at least one heteroatom se-lected from N, O and S,

where the substituents are selected from the group consisting of: F, C1, Br, OH, CN, NH2 and C1-C10-alkyl;

A is a bridging group selected from the group unsubstituted or at least mono-substituted N, P, C1-C6-alkane, C3-C10-cycloalkane, C3-C10-heterocycloalkane com-prising at least one heteroatom selected from N, O and S, C5-C14-aromatic and C5-C6-heteroaromatic comprising at least one heteroatom selected from N, O and S,

where the substituents are selected from the group consisting of:

C1-C4-alkyl, phenyl, F, C1, Br, OH, OR15, NH2, NHR15 or N(R15)2,

where R15 is selected from C1-C10-alkyl and C5-C10-aryl;

Y1, Y2, Y3 are, independently of one another, a bond, unsubstituted or at least monosubstituted methylene, ethylene, trimethylene, tetramethylene, pentamethylene or hexamethylene,

where the substituents are selected from the group consisting of: F, C1, Br, OH, OR15, CN, NH2, NHR15, N(R15)2 and C1-C10-alkyl,

where R15 is selected from C1-C10-alkyl and C5-C10-aryl.

In a further preferred embodiment, the process according to the invention is carried out in the presence of at least one complex catalyst which comprises at least one element selected from groups 8, 9 and 10 of the Periodic Table of the Elements and also at least one phosphorus donor ligand of the general formula (VIII), where

R7 to R10 are, independently of one another, methyl, ethyl, isopropyl, tert-butyl, cyclo-pentyl, cyclohexyl, phenyl, or mesityl;

A is

i) a bridging group selected from the group methane, ethane, propane, butane, cyclohexane, benzene, napthalene and anthracene;

or

ii) a bridging group of the formula (X) or (XI):

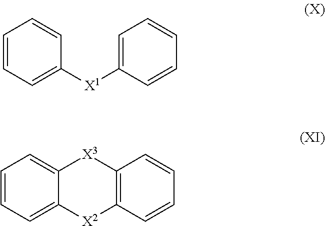

X1, X2 are, independently of one another, NH, 0 or S;

X3 is a bond, NH, O, S or CR17R18;

R17, R18 are, independently of one another, unsubstituted C1-C10-alkyl;

Y1, Y2 are, independently of one another, a bond, methylene or ethylene.

In a particularly preferred embodiment, the process according to the invention is carried out in the presence of at least one complex catalyst which comprises at least one element selected from groups 8, 9 and 10 of the Periodic Table of the Elements and also at least one phosphorus donor ligand of the general formula (XII) or (XIII),

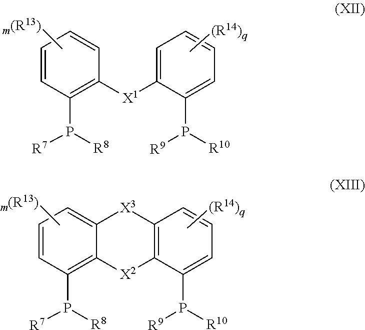

where for m, q, R7, R8, R9, R10, R13, R14, X1, X2 and X3, the definitions and preferences listed above are applicable.

In an embodiment, the process according to the invention is carried out in the presence of at least oneRu metal complex catalyst and monodentate ligands of the formula I are preferred herein are those in which R5a, R5b and R6 are each phenyl or alkyl optionally carrying 1 or 2 C1-C4-alkyl substituents and those in which R7, R8 and R9 are each C5-C8-cycloalkyl or C2-C10-alkyl, in particular linear unbranched n-C2-C10-alkyl. The groups R5a to R6 may be differ ent or identical. Preferably the groups R5a to R6 are identical and are selected from the substituents mentioned herein, in particular from those indicated as preferred. Examples of prefer able mono-dentate ligands IV are triphenylphosphine (TPP), Triethylphosphine, tri-n-butylphosphine, tri-n-octylphosphine and tricyclohexylphosphine.

In another embodiment, the process according to the invention is carried out in the presence of at least one Ru metal complex catalyst and at least one phosphorus donor ligand selected from the group consisting of 1,2-bis(diphenylphosphino)ethane (dppe), 1,2-bis(diphenylphosphino)propane (dppp), 1,2-bis(diphenylphosphino)butane (dppb), 2,3-bis(dicyclohexylphosphino)ethane (dcpe), 4,5-bis(diphenylphosphino)-9,9-dimethylxanthene (xantphos), bis(2-diphenylphosphinoethyl)phenylphosphine and 1,1,1-tris(diphenylphosphinomethyl)ethane (triphos).

In a further particularly preferred embodiment, the process according to the invention is carried out in the presence of a complex catalyst which comprises ruthenium and at least one phosphorus donor ligand selected from the group 4,5-bis(diphenylphosphino)-9,9-dimethylxanthene (xantphos), bis(2-diphenylphosphinoethyl)phenylphosphine and 1,1,1-tris(diphenylphosphinomethyl)ethane (triphos).

In a further particularly preferred embodiment, the process according to the invention is carried out in the presence of a complex catalyst which comprises iridium and also at least one phosphorus donor ligand selected from the group 4,5-bis(diphenylphosphino)-9,9-dimethylxanthene (xantphos), bis(2-diphenylphosphino¬ethyl)phenylphosphine and 1,1,1-tris(diphenylphosphinomethyl)ethane (triphos).

Within the context of the present invention, C1-C10-alkyl is understood as meaning branched, unbranched, saturated and unsaturated groups. Preference is given to alkyl groups having 1 to 6 carbon atoms (C1-C6-alkyl). More preference is given to alkyl groups having 1 to 4 carbon at oms (C1-C4-alkyl).

Examples of saturated alkyl groups are methyl, ethyl, n-propyl, isopropyl, n-butyl, isobutyl, secbutyl, tert-butyl, amyl and hexyl.

Examples of unsaturated alkyl groups (alkenyl, alkynyl) are vinyl, allyl, butenyl, ethynyl and propynyl.

The C1-C10-alkyl group can be unsubstituted or substituted with one or more substituents selected from the group F, C1, Br, hydroxy (OH), C1-C10-alkoxy, C5-C10-aryloxy, C5-C10-alkylaryloxy, C5-C10-heteroaryloxy comprising at least one heteroatom selected from N, O, S, oxo, C3-C10-cycloalkyl, phenyl, C5-C10-heteroaryl comprising at least one heteroatom selected from N, O, S, C5-C10-heterocyclyl comprising at least one heteroatom selected from N, O, S, naphthyl, amino, C1-C10-alkylamino, C5-C10-arylamino, C5-C10-heteroarylamino comprising at least one heteroatom selected from N, O, S, C1-C10-dialkylamino, C10-C12-diarylamino, 010-C20-alkylarylamino, C1-C10-acyl, C1-C10-acyloxy, NO2, C1-C10-carboxy, carbamoyl, carboxamide, cyano, sulfonyl, sulfonylamino, sulfinyl, sulfinylamino, thiol, C1-C10-alkylthiol, C5-C10-arylthiol or C1-C10-alkylsulfonyl.

The above definition for C1-C10-alkyl applies correspondingly to C1-C30-alkyl and to C1 C6 alkane.

C3-C10-cycloalkyl is understood in the present case as meaning saturated, unsaturated monocyclic and polycyclic groups. Examples of C3-C10-cycloalkyl are cyclopropyl, cyclobutyl, cyclopentyl, cyclohexyl or cycloheptyl. The cycloalkyl groups can be unsubstituted or substituted with one or more substituents as has been defined above in connection with the group C1-C10-alkyl.

The active vinylation catalyst can be generated in situ in the reaction mixture by adding the ligands to the above-mentioned precursors. The molar ratio between the transition metal and the ligand is in the range of 2:1 to 1:50, preferable in the range of 1:1 to 1:10 most preferably in the range of 1:2 to 1:5.

In addition to the one or more ligands selected from the groups of ligands described above the catalytic system of the inventive process may also include at least one further ligand which is selected from halides, amides, carboxylates, acetylacetonate, aryl- or alkylsufonates, hydride, CO, olefins, dienes, cycloolefins, nitriles, aromatics and heteroaromatics, ethers, PF3, phospholes, phosphabenzenes, and mono-, di- and polydentate phosphinite, phosphonite, phosphoramidite and phosphite ligands. Preferably the catalyst also contains CO as a ligand.

The active catalyst RuCat can also be preformed in a dedicated synthetic step. Appropriate pre formed catalysts can be [Ru(PPh3)3(C0)(H)Cl], [Ru(PPh3)3(CO)C12], [Ru(PPh3)3(C0)(H)2], [Ru(binap)(Cl)2], [Ru(PMe3)4(H)2], [Ru(PEt3)4(H)2], [Ru(Pn-Pr3)4(H)2], [Ru(Pn-Bu3)4(H)2], [Ru(Pn-Octyl3)4(H)2], [Ru(Pn-Bu3)4(H)2], [Ru(PnOctyl3)4(H)2], [Ru(PPh3)3(C0)(H)Cl] and [Ru(PPh3)3(C0)(H)2], preferably [Ru(PEt3)4(H)2], [Ru(Pn-Bu3)4(H)2] and [Ru(Pn-Octyl3)4(H)2].

In one embodiment of the present invention, the inventive process is characterized in that the homogeneous transition RuCat is selected from the group consisting of [Ru(PPh3)3(C0)(H)Cl], [Ru(PPh3)3(CO)C12], [Ru(PPh3)3(C0)(H)2], [Ru(binap)(Cl)2], [Ru(PMe3)4(H)2], [Ru(PEt3)4(H)2], [Ru(Pn-Pr3)4(H)2], [Ru(Pn-Bu3)4(H)2], [Ru(Pn-Octyl3)4(H)2], [Ru(PnBu3)4(H)2], [Ru(PnOctyl3)4(H)2], [Ru(PPh3)3(C0)(H)Cl] and [Ru(PPh3)3(C0)(H)2], preferably [Ru(PPh3)3(C0)(H)Cl], [Ru(PPh3)3(CO)C12] and [Ru(PPh3)3(C0)(H)2.

If a preformed active catalyst is used, it can also be beneficial to add additional ligand of the formula I or II to the reaction mixture.

In the inventive process the amount of RuCat used based on the cyclic compound C can be varied in a wide range. Usually RuCat is used in a sub-stoichiometric amount relative to the cyclic compound C. Typically, the amount of RuCat is not more than 50 mol %, frequently not more than 20 mol % and in particular not more than 10 mol % or not more than 5 mol %, based on the amount of the cyclic compound C. An amount of RuCat of from 0.001 to 50 mol %, frequently from 0.001 mol % to 20 mol % and in particular from 0.005 to 5 mol %, based on the amount of the cyclic compound C is preferably used in the process of the invention. Preference is given to using an amount of RuCat of from 0.01 to 5 mol %. All amounts of RuCat indicated are calculated as Ru metal and based on the amount of the cyclic compound C.

In one embodiment of the present invention, the inventive process is characterized in that the homogeneous RuCat is used in an amount of 0.001 mol % to 20 mol %, calculated as Ru metal and based on the amount of the cyclic compound C used in the process.

The reaction of a cyclic compound C with acetylene can principally be performed according to all processes known to a person skilled in the art which are suitable for the reaction of a cyclic compound C with acetylene.

The acetylene used for the reduction reaction can be used in pure form or, if desired, also in the form of mixtures with other, preferably inert gases, such as nitrogen or argon. Preference is given to using acetylene in undiluted form.

The acetylene can be applied discontinuously or continuously, e.g. by bubbling acetylene gas through the reaction mixture.

The reaction is typically carried at a acetylene pressure in the range from 0.1 to 10 bar, preferably in the range from 1 to 5 bar, more preferably in the range from 1 to 1.5 bar cold pressure.

In one embodiment of the present invention, the inventive process is characterized in that the reaction between a cyclic compound C and acetylene is performed at a pressure in the range from 1 to 15 bar.

The reaction can principally be performed continuously, semi-continuously or discontinuously. Preference is given to a continuous process.

The vinylation reaction according to the invention is carried out in a liquid phase. This can be achieved by adding one or more solvents, preferably from the group of aliphatic as well as aromatic hydrocarbons, linear as well as cyclic ethers, linear as well as cyclic amides, sulfoxides, nitriles and halogenated hydrocarbons. Preferred solvents are toluene, DMF and Di-glyme. The liquid phase can also be formed by the liquid cyclic compound C without any additional solvent.

Also one or more bases such as nitrogen bases like trialkylamines or pyridines, preferably N,N-dimethylaminopyridine can be added to the liquid phase, preferably in an amount of 0.5 to 20 equivalents according the amount of the used catalysts RuCat.

The reaction can principally be performed in all reactors known to a person skilled in the art for this type of reaction and who will therefore select the reactors accordingly. Suitable reactors are described and reviewed in the relevant prior art e.g. K. Henkel, “Reactor Types and Their Industrial Applications”, Ullmann's Encyclopedia of Industrial Chemistry, 2005, Wiley-VCH Verlag GmbH & Co. KGaA, chapter 3.3: “Reactors for gas-liquid reactions”.

The inventive process can be performed in a wide temperature range. Preferably the reaction is performed at a temperature in the range from 20° C. to 200° C., more preferably in the range from 50° C. to 180° C., in particular in the range from 100° C. to 170° C.

### EXAMPLES

A) General procedure for examples 1, 9, 10, 11, 12, 14, 18, 21, 22, 23, 25, 27, 29, 30, 31, 32, 33, 38, 40, 42: An approximately 40 mL autoclave (Premex, Hastelloy) was charged with CodRu(met)2 (0,001-0.06 mmol), cyclic compound C (1 mmol), toluene (5.0-10.0 mL) or dichloromethane (10 ml, entry 9) or dimethylformamide (5 ml, entries 20, 36, 37, 40, 41), and tri-n-butylphosphine (0,005-0.18 mmol) or trioctylphosphine (0.1 mmol, entry 12) or tricyclohexylphosphine (0.1 mmol, entry 13) under argon atmosphere in the Glove-box. After closing the reaction vessel, the system was purged with acetylene (3 times). Finally, the autoclave was pressurized with acetylene (at 1,5 bar for 15 min at room temperature) and heated at 100-140° C. The mixture was then stirred at the specified temperature for 14-18 h. Note: At this temperature the internal pressure rises to 3-4 bar. Then, the reaction was cooled down on a water bath and depressurized carefully. The crude mixture was collected in a round bottom flask and concentrated under vacuum. Subsequently, it was dissolved in 1 mL of CH2Cl2 and coated on silica. The product was isolated by column chromatography (petroleum ether/ethyl: acetate 8/2—for different products the system ratio was slightly varied). When the reaction was performed in DMF, the product was either extracted with dichloromethane from aqueous solution of the reaction mixture, organic layer was washed with water minimum 5 times (entries 40, 41) or collected in a round bot-tom flask and concentrated under vacuum followed by column chromatography isolation (petroleum ether/ethyl: acetate, entries 20, 36, 37).

B) General procedure for examples 13, 15, 16, 17, 19, 20, 26, 28, 34, 35, 36, 37, 39, 41: An approximately 40 mL autoclave (Premex, Hastelloy) was charged with CodRu(met)2 (0.02 0.06 mmol), cyclic compound C (1 mmol), toluene (5.0 mL, entries 15, 17, 18, 19, 21, 22, 33, 35, 43, 47, 49) or dimethylphormamide (5-8 mL, entries 42, 44, 45), DMAP (0.04-0.12 mmol) and trin-butylphosphine (0.06-0.18 mmol) under argon atmosphere in the Glovebox. After closing the reaction vessel, the system was purged with acetylene (3 times). Finally, the autoclave was pressurized with acetylene (at 1,5 bar for 15 min at room temperature) and heated at 100-150° C. The mixture was then stirred at the specified temperature for 14-18 h. Note: At this temperature the internal pressure rises to 4-6 bar. Then, the reaction was cooled down on a water bath and depressurized carefully. The crude mixture was collected in a round bottom flask and concentrated under vacuum. Subsequently, it was dissolved in 1 mL of CH2Cl2 and coated on silica. The product was isolated by column chromatography (petroleum ether/ethyl: acetate 8/2—for different products the system ratio was slightly varied).

C) General procedure for the comparative examples 2 and 3: An approximately 40 mL auto clave (Premex, Hastelloy) was charged with Ruthenium 5% on activated charcoal (100 mg), 2-pyrrolidinone (13.1 mmol, 1.116 g), and diglyme (for entry 13) or toluene (for entry 14) (8.0 mL) under argon atmosphere in the Glovebox. After closing the reaction vessel, the system was purged with acetylene (3 times). Finally, the autoclave was pressurized with acetylene (at 1,5 bar for 15 min at room temperature) and heated at 170° C. The mixture was then stirred at the specified temperature for 14 h. Note: At this temperature the internal pressure rises to 5 (diglyme)/7 (toluene) bar. Then, the reaction was cooled down on a water bath and depressurized carefully. The crude mixture was collected in a round bottom flask and concentrated under vacuum. The crude mixture was analyzed by GC and/or NMR. Product was not isolated. The comparative examples 2 and 3 show, that by using a heterogeneous Ru-catalyst only gives the de sired product in minor amounts.

D) General procedure for comparative example 4: An approximately 40 mL autoclave (Premex, Hastelloy) was charged with Ruthenium 5% on activated charcoal (10 mg), 2-pyrrolidinone (1 mmol, 0,085 g), and toluene (10.0 mL) under argon atmosphere in the Glovebox. After closing the reaction vessel, the system was purged with acetylene (3 times). Finally, the autoclave was pressurized with acetylene (at 1,5 bar for 15 min at room temperature) and heated at 170° C. The mixture was then stirred at the specified temperature for 14 h. Note: At this temperature the internal pressure rises to 7 bar. Then, the reaction was cooled down on a water bath and depressurized carefully. The crude mixture was collected in a round bottom flask and concentrated under vacuum. The crude mixture was analyzed by GC and/or NMR. Product was not isolated. The comparative example 4 shows, that by using a heterogeneous Ru-catalyst only gives the desired product in minor amounts.

E) General procedure for the comparative examples 5, 6, 7 and 8: An approximately 40 mL autoclave (Premex, Hastelloy) was charged with 2 mol % Ruthenium catalyst (RuCl3.3H2O entry 5; Ru(AcAc)3-entry 6; Ru3(CO)12-entry 7; codRumet2-entry 8), 2-pyrrolidinone (1 mmol, 0,085 g), and toluene (5.0 mL) under argon atmosphere in the Glovebox. After closing the reaction vessel, the system was purged with acetylene (3 times). Finally, the autoclave was pressurized with acetylene (at 1,5 bar for 15 min at room temperature) and heated at 100° C. The mixture was then stirred at the specified temperature for 16-19 h. Note: At this temperature the internal pressure rises to 3-4 bar. Then, the reaction was cooled down on a water bath and depressurized carefully. The crude mixture was collected in a round bottom flask and concentrated under vacuum. The crude mixture was analyzed by GC. Product was not isolated. The comparative examples 5, 6, 7 and 8 show, that by using only a Ruthenium complex without a phosphine ligand, under the same conditions only trace amounts of the desired product are formed.

F) General procedure for the comparative example 43: An approximately 40 mL autoclave (Premex) was charged with 20 mol % of tri-n-butylphosphine (0.2 mmol, 0,042 g), 2-pyrrolidinone (1 mmol, 0,085 g), and toluene (10.0 mL) under argon atmosphere in the Glovebox. After closing the reaction vessel, the system was purged with acetylene (3 times). Finally, the autoclave was pressurized with acetylene (at 1,5 bar for 15 min at room temperature) and heated at 100° C. The mixture was then stirred at the specified temperature for 16 h. Note: At this temperature the internal pressure rises to 3-4 bar. Then, the re-action was cooled down on a water bath and depressurized carefully. The crude mixture was collected in a round bottom flask and concentrated under vacuum. The crude mixture was analyzed by GC. Product was not formed. The comparative examples 43 shows, that by using only the phosphine ligand without a Ruthenium complex gives no product under these conditions.

G) General procedure for the example 24: An approximately 40 mL autoclave (Premex, Hastelloy) was charged with anhydrous ruthenium (Ill) chloride (0.03 mmol, entry 26) or ruthenium (Ill) chloride hydrate (0.03-0.09 mmol, entries 27, 29, 30, 31), 5-methyl-1,3-oxazolidin-2-one (1 mmol, 0,101 g), toluene (5.0 mL), and tri-n-butylphosphine (0,06-0.12 mmol, entries 26, 27, 29, 30) or triphenylphosphine (0.1 mmol, entry 31) under argon atmosphere in the Glovebox. After closing the reaction vessel, the system was purged with acetylene (3 times). Finally, the autoclave was pressurized with acetylene (at 1,5 bar for 15 min at room temperature) and heated at 100° C. The mixture was then stirred at the specified temperature for 14-16 h. Note: At this temperature the internal pressure rises to 3-4 bar. Then, the reaction was cooled down on a water bath and depressurized carefully. The crude mixture was collected in a round bottom flask and concentrated under vacuum. The crude mixture was analyzed by GC and/or NMR. Product was not isolated.

| | | | | | | | | | | | Conver- |
|---|---|---|---|---|---|---|---|---|---|---|---|
| | | | | | | | | | | | sion to |
| | | | | | | | | | | | product |
| | | | | | | | | | | | (% in |
| | | | | | | | | | | | reaction |
| | | | | | | | | | | | mixture |
| | | | | | | | | | | | de- |
| | Pro- | | | | | | | | | | termined |
| Ex- | ce- | Cyclic com- | | | | | T, | | | Yield, % | by |
| ample | dure | pound C | Cat, mol % | L1, mol % | L2, mol % | Solvent | ° C. | t, h | product | (isolated) | GC) |
| 1 | A | 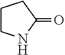 | codRumet₂/2 | P(nBu)₃/ 10 | — | Tol | 100 | 15 | 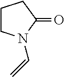 | 89 | 100 |
| 2 | C | | Ru/C/5 | — | — | Diglyme | 170 | 14 | | — | 6 |
| 3 | C | | Ru/C/5 | — | — | Tol | 170 | 16 | | — | 10 |
| 4 | D | | Ru/C/5 | — | — | Tol | 170 | 16 | | — | <1 |
| 5 | E | | RuCl₃—3H₂O/2 | — | — | Tol | 100 | 19 | | — | 5 |
| 6 | E | | Ru(AcAc)₃/2 | — | — | Tol | 100 | 19 | | — | 0 |
| 7 | E | | Ru₃(CO)₁₂/2 | — | — | Tol | 100 | 19 | | — | 8 |
| 8 | E | | codRumet₂/2 | — | — | Tol | 100 | 16 | | — | 0, 9 |
| 9 | A | | codRumet₂/1 | P(nBu)₃/ 10 | — | Tol | 100 | 17 | | 78 | |
| 10 | A | | codRumet₂/0, 1 | PnBu₃/ 0, 5 | — | Tol | 100 | 17 | | 26 | |
| 11 | A | | codRumet₂/2 | P(OCt)₃/ 10 | — | Tol | 100 | 17 | | 79 | |
| 12 | A | | codRumet₂/2 | PCy₃ | — | Tol | 100 | 17 | | 35 | |
| 13 | B | 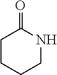 | codRumet₂/2 | P(nBu)₃/ 6 | DMAP/4 | Tol | 100 | 6 | 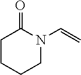 | 85 | 100 |
| 14 | A | 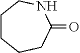 | codRumet₂/2 | P(nBu)₃/ 10 | — | Tol | 100 | 17, 5 | 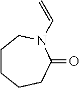 | 33 | 33 |
| 15 | B | | codRumet₂/2 | P(nBu)₃/ 10 (6) | DMAP/4 | Tol | 100 | 16 | | 80 | 100 |
| 16 | B | 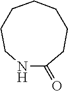 | codRumet₂/2 | P(nBu)₃/ 6 | DMAP/4 | Tol | 100 | 14, 5 | 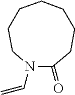 | 60 | 74 |
| 17 | B | 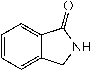 | codRumet₂/2 | P(nBu)₃/ 6 | DMAP/4 | Tol | 150 | 15 | 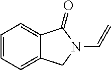 | 75 | 100 |
| 18 | A | 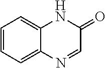 [SMILES: O=c1cnc2ccccc2[nH]1] | codRumet₂/4 | P(nBu)₃/ 6 | — | DMF | 140 | 17 | 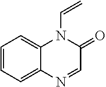 | 13 | Nd |
| 19 | B | 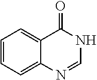 [SMILES: O=c1[nH]cnc2ccccc12] | codRumet₂/2 | P(nBu)₃/ 6 | DMAP/4 | Tol | 140 | 16 | 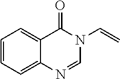 | 58 | nd |
| 20 | B | 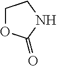 | codRumet₂/2 | P(nBu)₃/ 6 | DMAP/4 | Tol | 100 | 16 | 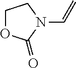 | 9 | 100 |
| 21 | A | | codRumet₂/2 | P(nBu)₃/ 6 | — | Tol | 100 | 16 | | 29 | 100 |
| 22 | A | 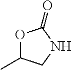 | codRumet₂/2 | P(nBu)₃/ 10 | — | Tol | 100 | 16 | 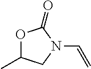 | 84 | 100 |
| 23 | A | | codRumet₂/2 | PPh₃/ 10 | — | Tol | 100 | 16 | | 17 | 20 |
| 24 | G | | RuCl₃—H₂O/9 | P(nBu)₃/ 12 | — | Tol | 100 | 14.5 | | nd | 100 |
| 25 | A | 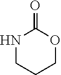 | codRumet₂/2 | P(nBu)₃/ 6 | — | Tol | 100 | 16 | 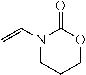 | 82.2 | 100 |
| 26 | B | 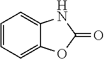 | codRumet₂/2 | P(nBu)₃/ 6 | DMAP/4 | Tol | 100 | 16 | 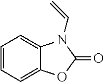 | 10 | nd |
| 27 | A | | codRumet₂/2 | P(nBu)₃/ 10 | — | Tol | 100 | 15 | | 25 | nd |
| 28 | B | 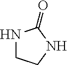 | codRumet₂/2 | P(nBu)₃/ 6 | DMAP/4 | Tol | 120 | 16 | 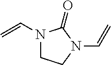 | 15 | nd |
| 29 | A | 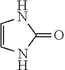 | codRumet₂/2 | P(nBu)₃/ 6 | — | DMF | 140 | 16 | 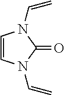 | 25 | 57 |
| 30 | A | 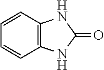 | codRumet₂/6 | P(nBu)₃/ 18 | — | DMF | 140 | 16 | 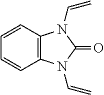 | 27 | nd |
| 31 | A | 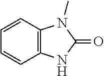 | codRumet₂/2 | P(nBu)₃/ 6 | — | Tol | 130 | 16 | 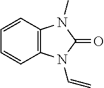 | 37 | nd |
| 32 | A | 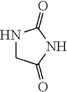 | codRumet₂/2 | P(nBu)₃/ 10 | — | DMF | 100 | 16 | 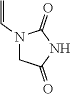 | 56 | nd |
| 33 | A | 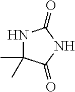 | codRumet₂/2 | P(nBu)₃/ 10 | — | DMF | 100 | 18 | 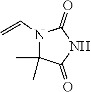 | 38 | nd |
| 34 | B | | codRumet₂/6 | P(nBu)₃/ 18 | DMAP/1 2 | DMF | 130 | 18 | | 30 | nd |
| 35 | B | 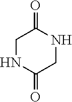 | codRumet₂/2 | P(nBu)₃/ 6 | DMAP/4 | Tol | 150 | 14 | 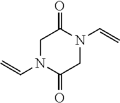 | 14 | nd |
| 36 | A | 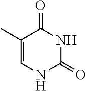 | codRumet₂/2 | P(nBu)₃/ 6 | DMAP/4 | DMF | 150 | 16 | 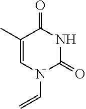 | 20 | nd |
| 37 | B | | codRumet₂/6 | P(nBu)₃/ 18 | DMAP/1 2 | DMF | 150 | 16 | 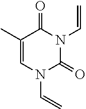 | 11 | nd |
| 38 | A | 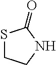 | codRumet₂/2 | P(nBu)₃/ 6 | — | Tol | 100 | 15 | 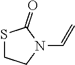 | 22 | 64 |
| 39 | B | | codRumet₂/2 | P(nBu)₃/ 6 | DMAP/4 | Tol | 100 | 15 | | nd | 82 |
| 40 | A | | codRumet₂/2 | P(nBu)₃/ 10 | — | Tol | 100 | 15 | | nd | 51 |
| 41 | B | | codRumet₂/3 | P(nBu)₃/ 6 | DMAP/4 | Tol | 100 | 15 | | 49 | 97 |
| 42 | A | 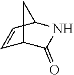 | codRumet₂/2 | P(nBu)₃/ 10 | — | Tol | 100 | 16 | 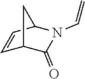 | 57 | 82 |
| 43 | F | 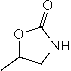 | — | P(nBu)₃/ 20 | — | Tol | 100 | 16 | 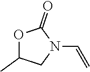 | 0 | 0 |

1. A process to produce N-vinyl compounds by homogeneous catalysis, the process comprising:
reacting acetylene with a cyclic compound C having at least one nitrogen as a ring member, bearing a substitutable hydrogen residue, in a liquid phase in the presence of a ruthenium complex comprising at least one phosphine as a ligand (RuCat).

2. The process according claim 1, wherein the cyclic compound C is a cyclic amide, a cyclic urea or thiourea, or a cyclic carbamate or thiocarbamate.

3. The process according to claim 1, wherein the cyclic compound C is a cyclic amide.

4. The process according to claim 1, wherein the at least one phosphine is a mono-, di-, tri- or tetra dentate phosphine.

5. The process according to claim 1, wherein the at least one phosphine is a phosphine of formula (I) or (II), 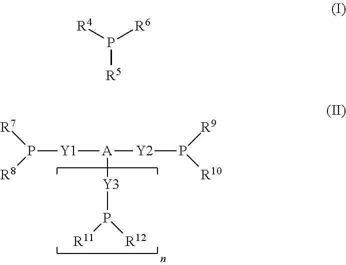
wherein
n is 0 or 1;
R⁴ to R¹² are, independently of one another, unsubstituted or at least monosubstituted C₁-C₁₀-alkyl, C₃-C₁₀ cycloalkyl, C₃-C₁₀-heterocyclyl comprising at least one heteroatom selected from the group consisting of N, O, and S, C₅-C₁₄-aryl or C₅-C₁₀-heteroaryl comprising at least one heteroatom selected from the group consisting of N, O, and S, where substituents are selected from the group consisting of: F, Cl, Br, OH, CN, NH₂ and C₁-C₁₀ alkyl;
A is
i) a bridging group selected from the group consisting of unsubstituted or at least monosubstituted N, O, P, C₁-C₆-alkane, C₃-C₁₀-cycloalkane, C₃-C₁₀-heterocycloalkane comprising at least one heteroatom selected from the group consisting of N, O, and S, C₅-C₁₄-aromatic, and C₅-C₁₀-heteroaromatic comprising at least one heteroatom selected from the group consisting of N, O and S, where substituents are selected from the group consisting of C₁-C₄-alkyl, phenyl, F, Cl, Br, OH, OR¹⁶, NH₂, NHR¹⁶, and N(R¹⁶)₂,
where R¹⁶ is selected from the group consisting of C₁-C₁₀-alkyl and C5-C₁₀-aryl;
or
ii) a bridging group of the formula (VI) or (VII): 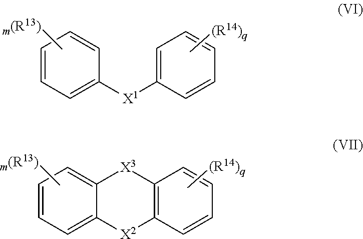
wherein
m, q are, independently of one another, 0, 1, 2, 3 or 4;
R¹³, R¹⁴ are, independently of one another, selected from the group consisting of C₁-C₁₀-alkyl, F, Cl, Br, OH, OR¹⁵, NH₂, NHR¹⁵ and N(R¹⁵)₂,
where R¹⁵ is selected from the group consisting of C₁-C₁₀-alkyl and C₅-C₁₀-aryl;
X¹, X² are, independently of one another, NH, O or S;
X³ is a bond, NH, NR¹⁶, O, S or CR¹⁷R¹⁸,
R¹⁶ is unsubstituted or at least monosubstituted C₁-C₁₀-alkyl, C₃-C₁₀-cycloalkyl, C₃-C₁₀-heterocyclyl comprising at least one heteroatom selected from the group consisting of N, O and S, C₅-C₁₄-aryl or C₅-C₁₀-heteroaryl comprising at least one heteroatom selected from the group consisting of N, O and S, where substituents are selected from the group consisting of F, Cl, Br, OH, CN, NH₂ and C₁-C₁₀-alkyl;
R¹⁷, R¹⁸ are, independently of one another, unsubstituted or at least monosubstituted C₁-C₁₀-alkoxy, C₁-C₁₀-alkoxy, C₃-C₁₀-cycloalkyl, C₃-C¹⁰ cycloalkoxy, C₃-C₁₀-heterocyclyl comprising at least one heteroatom selected from the group consisting of N, O and S, C₅-C₁₄-aryl, C₅-C₁₄-aryloxy or C₅-C₁₀-heteroaryl comprising at least one heteroatom selected from the group consisting of N, O, and S, where substituents are selected from the group consisting of F, Cl, Br, OH, CN, NH₂, and C₁-C₁₀-alkyl;
Y¹, Y², Y³ are, independently of one another, a bond, unsubstituted or at least monosubstituted methylene, ethylene, trimethylene, tetramethylene, pentamethylene or hexamethylene, where substituents are selected from the group consisting of F, Cl, Br, OH, OR¹⁵, CN, NH₂, NHR¹⁵, N(R¹⁵)₂, and C₁-C₁₀-alkyl,
where R¹⁵ is selected from the group consisting of C₁-C₁₀-alkyl and C5-C10-aryl.

6. The process according to claim 5, wherein the at least one phosphine is a phosphine of formula (I).

7. The process according to claim 6, wherein the at least one phosphine is a trialkyl phosphine.

8. The process according to claim 1, wherein the ruthenium complex is prepared separately or in situ during the reaction of the cyclic compound C with acetylene.

9. The process according to claim 8, wherein 1 to 10 mol of phosphine per mol of ruthenium are used in the preparation of the ruthenium complex.

10. The process according to claim 1, wherein the ruthenium complex is used in an amount of 0.01 to 5 mot % based on an amount of the cyclic compound C.

11. The process according to claim 1, wherein the reaction of the cyclic compound C and the acetylene is performed in presence of a N-base.

12. The process according to claim 1, wherein the liquid phase comprises a solvent.

13. The process according to claim 1, wherein the acetylene is fed to the reaction with a pressure of 1 to 2 bar at 20° C.

14. The process, according to claim 1, wherein the reaction is performed at a temperature of 50 to 200° C.

15. The process according to claim 1, wherein a pressure during the reaction is at maximum 10 bar.
